(19) United States
(12) Patent Application Publication
(10) Pub. No.: US 2026/0013950 A1
(43) Pub. Date: Jan. 15, 2026

(54) METHODS, SYSTEMS, AND GUI FOR ENHANCED REAL-TIME VISUAL FEEDBACK OF INTRALUMINAL CATHETER ENGAGEMENT

(71) Applicant: BIOSENSE WEBSTER (ISRAEL) LTD., Yokneam (IL)

(72) Inventors: NATAN SHARON KATZ, ATLIT (IL); BENJAMIN COHEN, HAIFA (IL); DROR BERMAN, IRVINE (IL); RONI ROSENZWEIG, HAIFA (IL)

(21) Appl. No.: 19/225,052

(22) Filed: Jun. 2, 2025

(30) Foreign Application Priority Data
Jul. 11, 2024 (IL) 314268

(51) Int. Cl. A61B 34/20; A61B 18/00; A61B 18/14; A61B 34/00

(52) U.S. Cl. CPC A61B 34/20; A61B 18/1492; A61B 34/25; A61B 2018/00351; A61B 2018/00577; A61B 2018/00875

(57) ABSTRACT

The presently disclosed subject matter includes a computer system, method, and graphical user interface that provide graphical feedback indicating real-time contact level between a tissue wall of a luminal organ and an ablation catheter, based on electrical impedance sensed by electrodes of a catheter. As the impedance is related to proximity of the electrodes to the tissue walls, the system utilizes impedance measurements to visually alter the appearance of a graphical representation of the electrodes according to changes in the sensed impedance. The graphical feedback enables the physician to make real-time adjustments to the catheter's positioning and applied force, and thereby enhance the precision and effectiveness of intraluminal catheter therapy. The graphical feedback comprises a plurality of contact lobes centered on the electrodes and have sizes which increase with the contact level.

### TECHNOLOGICAL FIELD

The presently disclosed subject matter relates to visualization techniques for intraluminal catheter therapy in the diagnosis and treatment of medical disorders.

### BACKGROUND

Intraluminal catheter therapy (ICT) or catheterization has become a pivotal instrument in the field of medicine for both the diagnosis and treatment of various medical disorders. This minimally invasive procedure involves guiding a slender, flexible tube, or catheter, into a luminal organ. Equipped with electrodes, the catheter is used, inter alia, for mapping the organ, and identifying precise locations related to abnormal medical conditions.

Cardiac ICT, a particular example of ICT, is an important tool for both the diagnosis and treatment of cardiac disorders, particularly arrhythmias. This involves inserting a catheter equipped with electrodes through the blood vessels and into the heart, using the catheter for generating a map of the heart's electrical activity, and identifying the precise locations of abnormal electrical activity. The identified sites can then be treated through ablation, where targeted energy neutralizes the abnormal tissue, restoring normal heart rhythm. This integrated approach has revolutionized cardiac care, offering patients less invasive options with shorter recovery times.

### OVERVIEW

An important aspect of catheterization in the context of cardiology is related to tissue contact and ablation accuracy. Ensuring adequate contact between the catheter distal end assembly (e.g., balloon and/or basket) located at its distal end and the heart tissue is crucial for accurate and effective results.

A physician manipulating a catheter would benefit from knowing what a contact level of the catheter with a tissue wall is. In other words, how much force is being applied on the tissue wall. The force distribution on a distal end assembly can help the physician to accurately steer the end. Further, it is desired to avoid the application of excessive force on the tissue when manipulating the catheter, as this may result in deformation of the tissue surface, e.g., tenting, and consequently cause inefficient operation. An indication of force being applied on the tissue walls is also helpful during various other procedures such as Pulsed Field Ablation (PFA). Too little contact can render the ablation ineffective, while too much pressure can cause excessive tissue damage. An indication of the proximity between the catheter and tissue can help the physician reach a threshold level of force to achieve a desired lesion depth.

The presently disclosed subject matter includes a computer system, method, and graphical user interface that provide graphical feedback indicating real-time proximity based on electrical impedance sensed by electrodes of a catheter. As the impedance is related to proximity of the electrodes to the tissue walls, the system utilizes impedance measurements to visually alter appearance of a graphical representation of the electrodes according to changes in the sensed impedance. The graphical feedback enables the physician to make real-time adjustments to the catheter's positioning and applied force, and thereby enhance the precision and effectiveness of ICT. For instance, if the impedance suggests inadequate contact, the physician may reposition the catheter to achieve better engagement with the tissue. Conversely, a high impedance reading could signal excessive pressure, prompting the physician to reduce the contact force to prevent tenting and/or potential tissue damage.

A problem addressed by the current disclosure is achieving a balance between high-detail visualization and a sufficiently simple structure of visual elements. This issue is resolved in this disclosure by using ‘contact lobes’, formed by curves overlaid (superimposed) on the rendering of the catheter, and positioned at an optimal distance range. The size of these contact lobes indicates the level of contact. Additionally, the contact lobes are designed to be easily distinguishable from one another.

### BRIEF DESCRIPTION OF THE DRAWINGS

In order to better understand the subject matter that is disclosed herein and to exemplify how it may be carried out in practice, examples will now be described, by way of non-limiting example only, with reference to the accompanying drawings, in which:

FIG. 1A illustrates a catheter-based electrophysiology mapping and ablation system, according to examples of the presently disclosed subject matter;

FIG. 1B shows a flowchart illustrating a computer-implemented method according to embodiments of the present disclosure.

FIG. 1C schematically illustrates transverse and tangential directions, according to embodiments of the present disclosure.

FIG. 2A-2F schematically illustrate various contact lobes according to embodiments of the present disclosure.

FIG. 3A-3B schematically illustrate merging of non-adjacent contact lobes, according to embodiments of the present disclosure.

FIG. 3C-3E schematically illustrate an inner side according to embodiments of the present disclosure.

FIG. 4A schematically illustrates transverse and tangential radii, according to embodiments of the present disclosure.

FIG. 4B schematically illustrates bending of contact lobes, according to embodiments of the present disclosure.

FIG. 5A-5D show exemplary screenshots of visual output of methods and systems according to embodiments of the present disclosure.

FIG. 6 shows a block diagram schematically illustrating a system according to embodiments of the present disclosure.

### DETAILED DESCRIPTION

In the following detailed description, numerous specific details are set forth in order to provide a thorough understanding. However, it will be understood by those skilled in the art that the presently disclosed subject matter may be practiced without these specific details. In other instances, well-known methods and features have not been described in detail so as not to obscure the presently disclosed subject matter.

Unless specifically stated otherwise, as apparent from the following discussions, it is appreciated that throughout the specification discussions utilizing terms such as “processing”, “computing”, “updating”, “rendering” or the like, refer to the action(s) and/or process(es) of a computer that manipulate and/or transform data into other data, said data represented as physical, such as electronic, quantities and/or said data representing the physical objects. The term “computer” should be expansively construed to cover any kind of hardware-based electronic device with data processing capabilities. Processing capabilities may include processing circuitry. Each processing circuitry can comprise, for example, one or more processors operatively connected to (including non-transitory) computer memory, loaded with executable instructions for executing operations, as further described below.

The one or more processors referred to herein can represent for example, one or more general-purpose processing devices such as a microprocessor, a central processing unit, or the like. More particularly, a given processor may be one of: a complex instruction set computing (CISC) microprocessor, a reduced instruction set computing (RISC) microprocessor, a very long instruction word (VLIW) microprocessor, processor implementing other instruction sets, or a processor implementing a combination of instruction sets. The one or more processors may also be one or more special-purpose processing devices such as an application specific integrated circuit (ASIC), a field programmable gate array (FPGA), a digital signal processor (DSP), a graphics processing unit (GPU), a network processor, or the like.

The various illustrative logical blocks, modules, and algorithm steps described in connection with the examples disclosed herein can be implemented as electronic hardware, computer software, or combinations of both. To clearly illustrate this interchangeability of hardware and software, various illustrative components, blocks, modules, and steps are described generally in terms of their functionality. Whether such functionality is implemented as hardware or software depends upon the particular application and design constraints imposed on the overall system. The described functionality can be implemented in varying ways for each particular application, but such implementation decisions should not be interpreted as causing any departure from the scope of the disclosure.

It will also be understood that a system according to the present disclosure may be, at least partly, implemented on a suitably programmed computer. Likewise, the present disclosure contemplates a computer program being readable by a computer for executing the method of the present disclosure. The present disclosure further contemplates a non-transitory computer-readable memory tangibly embodying a program of instructions executable by the computer for executing the method of the present disclosure.

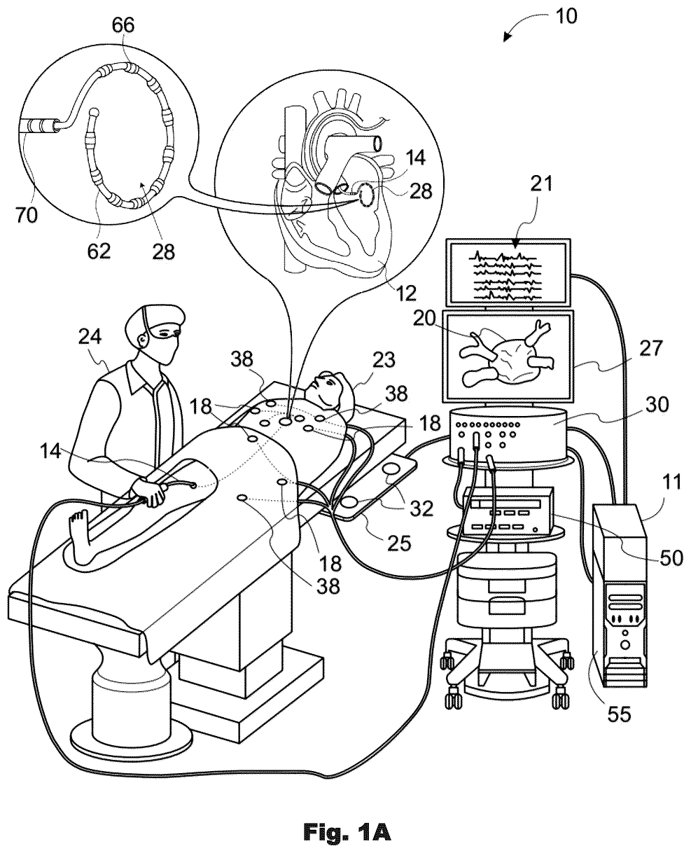

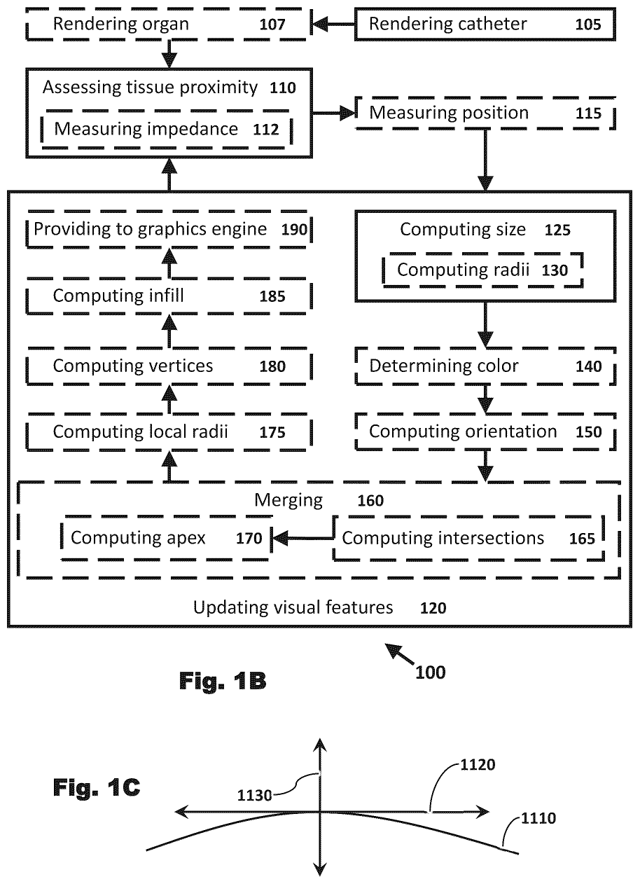

Reference is made to FIG. 1A showing an example catheter-based electrophysiology mapping and ablation system 10. System 10 includes multiple catheters, which are percutaneously inserted by a physician 24 through the patient's vascular system into a chamber or vascular structure of a heart 12. Typically, a delivery sheath catheter is inserted into the left or right atrium near a desired location in heart 12. Thereafter, the catheter may be inserted into the delivery sheath catheter so as to arrive at the desired location in the heart 12.

The plurality of catheters may include catheters dedicated for sensing Intracardiac Electrogram (IEGM) signals, catheters dedicated for ablating and/or catheters dedicated for both sensing and ablating. An example catheter 14 that is configured for ablating tissue and/or for sensing electrical cardiac activity and/or mapping is illustrated herein. Physician 24 may place a distal tip 28 of catheter 14 in contact with the epicardial tissue surface for sensing and/or ablating.

Catheter 14 is an exemplary catheter that includes a distal assembly, having one and preferably multiple electrodes 66 optionally distributed over a plurality of frame elements 62 at distal tip 28. The electrodes are generally configured for delivering ablation energy to tissue, and/or for sensing cardiac electrical signals. Catheter 14 additionally includes one or more position sensors 70 embedded in or near distal tip 28 for tracking position and orientation of distal tip 28. Optionally and preferably, position sensor 70 is a magnetic based position sensor, for example a position sensor including three magnetic coils for sensing three-dimensional (3D) position and orientation; or a position sensor including one magnetic coil, for sensing a single direction.

Each of the magnetic based position sensors 70 may be operated together with a location pad 25 including a plurality of magnetic coils 32 configured to generate magnetic fields in a predefined working volume. Real time position of distal tip 28 of catheter 14 may be tracked based on magnetic fields generated with location pad 25 and sensed by magnetic based position sensor 70. Details of the magnetic based position sensing technology are described in U.S. Pat. Nos. 5,5391, 199; 5,443,489; 5,558,091; 6,172,499; 6,239,724; 6,332,089; 6,484,118; 6,618,612; 6,690,963; 6,788,967; 6,892,091.

System 10 includes one or more electrode patches 38 positioned for skin contact on patient 23 to establish location reference for location pad 25 as well as impedance-based tracking of electrodes 66. For impedance-based tracking, electrical current is directed to electrodes 66 and sensed at electrode skin patches 38 so that the location of each electrode can be triangulated via the electrode patches 38. Details of the impedance-based location tracking technology are described in U.S. Pat. Nos. 7,536,218; 7,756,576; 7,848,787; 7,869,865; and 8,456,182.

A recorder 11 records and displays electrograms 21 captured with body surface ECG electrodes 18 and optionally cardiac signals captured with electrodes 66 of catheter 14. Recorder 11 may include pacing capability for pacing the heart rhythm and/or may be electrically connected to a standalone pacer.

System 10 may include an ablation energy generator 50 that is adapted to conduct ablative energy to electrodes 66 of the distal assembly at a distal tip of the catheter. Energy produced by ablation energy generator 50 may include, but is not limited to, radiofrequency (RF) energy or pulsed-field ablation (PFA) energy, including monopolar or bipolar high-voltage DC pulses as may be used to effect irreversible electroporation (IRE), or combinations thereof.

Patient interface unit (PIU) 30 is an interface configured to establish electrical communication between the at least one catheter with other catheters, other electrophysiological equipment, power supply, and a workstation 55 for controlling operation of system 10. Electrophysiological equipment of system 10 may include for example, multiple catheters, location pad 25, body surface ECG electrodes 18, electrode patches 38, ablation energy generator 50, and recorder 11. Optionally and preferably, PIU 30 additionally includes processing capability for implementing real-time computations of location of the catheters and for performing ECG calculations.

Workstation 55 includes memory, processor unit with memory or storage with appropriate operating software stored therein, and user interface capability. Workstation 55 may provide multiple functions, optionally including (1) modeling the endocardial anatomy in three-dimensions (3D) and rendering the model or anatomical map 20 for display on a display device 27, (2) displaying on display device 27 activation sequences (or other data) compiled from recorded electrograms 21 in representative visual indicia or imagery superimposed on the rendered anatomical map 20, (3) displaying real-time location and orientation of the at least one catheter within the heart chamber, and (4) displaying on display device 27 sites of interest such as places where ablation energy has been applied. One commercial product embodying elements of the system 10 is available as the CARTO™ 3 System, available from Biosense Webster, Inc., 31A Technology Drive, Irvine, CA 92618.

In some examples, an irrigation module is provided for delivering irrigation fluid, such as saline solution, to the location of treatment. The irrigation module may comprise a pump and an associated fluid tank.

Reference is made to FIG. 1B showing a flowchart illustrating a computer-implemented method 100 according to embodiments of the present disclosure. The method 100 may be used for providing real-time visual feedback. That is, providing visual feedback with negligible time delay. The visual feedback may be of a contact level between a tissue wall of a luminal organ and an ablation catheter (e.g., catheter 14 illustrated in FIG. 1A). The catheter may include a plurality of electrodes that may be placed along a catheter distal assembly. The plurality of electrodes may form an elongated electrode array.

In some embodiments, the plurality of electrodes may form a planar electrode array. For example, the catheter distal assembly may include a single surface on which electrodes may be disposed. In some embodiments, all electrodes included in the plurality of electrodes may form a linear array. For example, a single electrode may be included in each frame element.

The method 100 may be performed while the catheter distal assembly is in the luminal organ of a patient. In some embodiments, the luminal organ may be a heart of the patient, or a portion thereof.

The method 100 may include a step 105 of rendering on a display a graphical representation of the catheter distal assembly and the plurality of electrodes thereon. In some embodiments, the method 100 may include a step 107 of rendering on the display an anatomy of the luminal organ.

The method 100 may include a step 110 of repeatedly assessing tissue proximity. In some embodiments, the step 110 of repeatedly assessing tissue proximity may include a step 112 of repeatedly measuring impedance of at least one electrode. In other words, a predefined number of impedance measurements may be performed at a predefined time period. Preferably, impedance of each of the plurality of electrodes may be repeatedly measured.

In some embodiments, the method 100 may include a step 115 of measuring a position of the at least one electrode. Preferably, position of each of the plurality of electrodes may be measured. Step 115 of measuring the position of the at least one electrode may be required, for example, if the catheter was manipulated so the at least one electrode was moved from a known position. In some embodiments, step 115 of measuring the position may be performed before the step 110 of repeatedly measuring impedance of at least one electrode.

The method 100 may include a step 120 of dynamically updating visual features. The visual features may be indicative of the measured impedance. In other words, visual features may be updated according to the measured impedance. The updating may be performed immediately upon a measurement value being received. The updating may be performed each time an impedance measurement is received. The step 120 of dynamically updating visual features may include performing steps or routines according to the exact nature of the visual features. The routines and visual features are further described hereinbelow.

The visual features may include a plurality of contact lobes. Contact lobes may be line segments having visual properties indicative of the contact level between the tissue wall and the catheter distal assembly. The contact lobes may correspond to the plurality of electrodes along the catheter distal assembly. The contact lobes may be distinct. That is, the line segments forming the contact lobes may be configured so that each contact lobe may be easily distinguished from an adjacent contact lobe by a human watching a rendering of the contact lobes, such as a physician operating the catheter. It is noted that the contact lobes may form a continuous contour, as described herein below.

A size of a contact lobe may increase with the contact level between the tissue wall and the corresponding electrode, based on the measured impedance. For example, the contact lobes may have a geometry of circular arcs. The size of the contact lobes may be a radius of the circle on which the contact lobes may be based and/or a length of the arcs.

It is noted that the size of a contact lobe may be length of a specific geometrical feature of the contact lobe. In other words, the shape of the contact lobes may be variable. For example, the contact lobes may have a geometry of ellipsoidal arcs. Their size may be a length of one of the semi-major axis or the semi-minor axis. In some embodiments, one axis may be constant. That is, if the size is the length of the semi-major axis, the semi-minor axis may be constant. If the size is the length of the semi-minor axis, the semi-major axis may be constant.

Reference is made to FIG. 1C, schematically illustrating transverse and tangential directions, according to embodiments of the present disclosure. A catheter distal assembly 1110 may define a tangential direction 1120 and a transverse direction 1130. The tangential direction 1120 may be along a local orientation of the catheter distal assembly 1110. In other words, the tangential direction 1120 may be along a line tangent to a contour of the catheter distal assembly 1110. The transverse direction 1130 may be perpendicular to the tangential direction 1120.

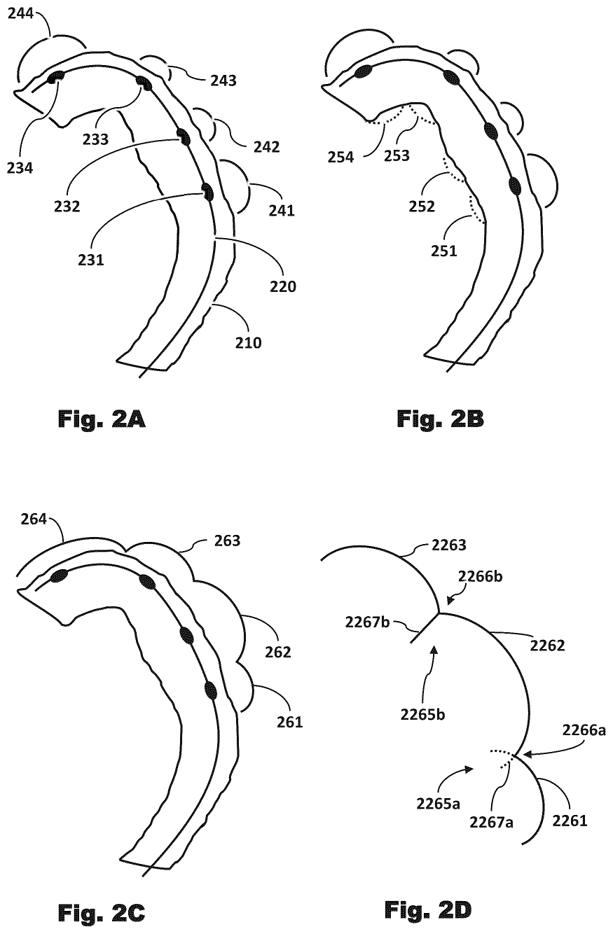

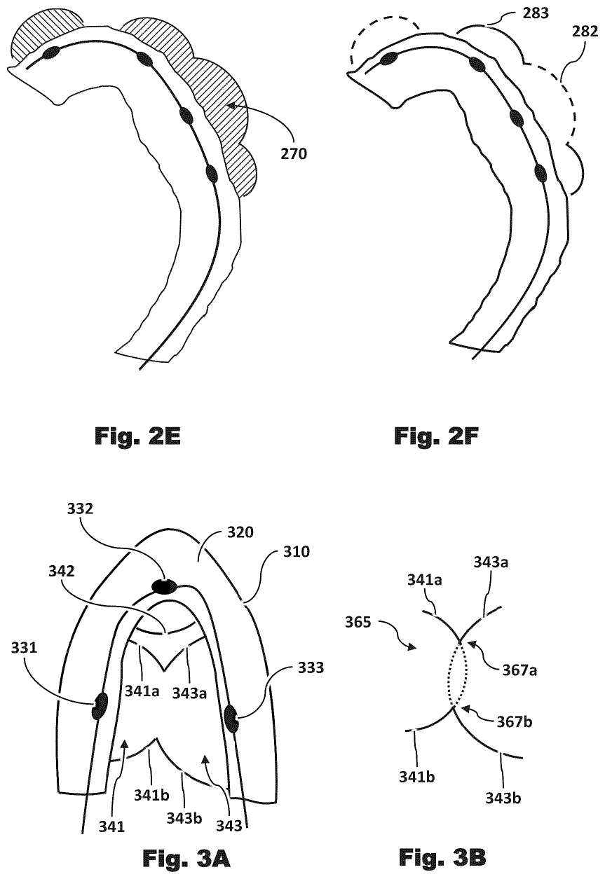

Reference is made to FIG. 2A, schematically illustrating exemplary contact lobes according to embodiments of the present disclosure. A catheter distal assembly 220 that may be positioned inside an intraluminal organ 210 may be rendered on a display. The catheter distal assembly 220 may include a plurality of electrodes 231, 232, 233, and 234. For each electrode, a corresponding contact lobe is rendered. That is, contact lobe 241 may correspond to electrode 231, contact lobe 242 may correspond to electrode 232, contact lobe 243 may correspond to electrode 233, and contact lobe 244 may correspond to electrode 234. The contact lobes 241, 242, 243, and 244 may be overlaid on the graphical representation of the catheter distal assembly 220.

The contact lobes 241, 242, 243, and 244 may each be centered on a corresponding electrode. That is, the line segments forming the contact lobes 241, 242, 243, and 244 may be configured so that each contact lobe may easily indicate the corresponding electrode by a human watching a rendering of the contact lobes, such as a physician operating the catheter. For example, the contact lobes 241, 242, 243, and 244 may have a symmetry axis. The symmetry axis may pass through the corresponding electrode. In some embodiments, the symmetry axis may pass nearby the corresponding electrode. In some embodiments, the symmetry axis may be parallel to the transverse direction of the distal assembly 220.

The contact lobes 241, 242, 243, and 244 may follow a contour of the graphical representation of the catheter distal assembly 220. In other words, for each of the contact lobes 241, 242, 243, and 244, an axis defining a longitudinal extent of the contact lobe may be parallel to the tangential direction the catheter distal assembly 220. For example, the contact lobes 241, 242, 243, and 244 may be ellipsoidal segments. In other words, the contact lobes 241, 242, 243, and 244 may have a geometry of ellipsoidal arcs. An axis of the ellipse (i.e., the major axis or the minor axis) may be parallel to the tangential direction of the catheter distal assembly 220.

The contact lobes 241, 242, 243, and 244 may be positioned on one side of the catheter distal assembly 220. The side of the catheter distal assembly 220 on which contact lobes 241, 242, 243, and 244 may be positioned, may indicate the side of the organ 210 which the plurality of electrodes 231, 232, 233, and 234 are in contact with. In other words, a line parallel to the transverse direction, connecting a point on a contact lobe to a corresponding electrode, may pass through the side of the organ 210 which the electrode is in contact with, but not through a side of the organ 210 which the electrode is not in contact with.

Reference is made to FIG. 2B schematically illustrating other exemplary contact lobes according to embodiments of the present disclosure. The embodiments illustrated in FIG. 2B add to the embodiments illustrated in FIG. 2A contact lobes 251, 251, 253, and 254. For brevity, reference numerals of elements already referred to in describing FIG. 2A are not shown in FIG. 2B. Contact lobe 251 corresponds to electrode 231, contact lobe 252 corresponds to electrode 232, contact lobe 253 corresponds to electrode 233, and contact lobe 254 corresponds to electrode 234. Contact lobes 251, 251, 253, and 254 may be positioned on a side of the catheter distal assembly 220 opposite to a side on which contact lobes 241, 242, 243, and 244 may be positioned.

In some embodiments, the contact lobes may be configured to expand asymmetrically with respect to a central axis of the catheter distal assembly, so as to predominantly extend in a transverse direction towards the tissue wall. In other words, contact lobes 241, 242, 243, and 244 may have a greater size than contact lobes 251, 251, 253, and 254.

As indicated herein above, the contact lobes may form a continuous contour. In other words, at least two contact lobes may intersect or may share a common point. Preferably, in some embodiments, all contact lobes may form a continuous contour. Reference is made to FIG. 2C, schematically illustrating yet other exemplary contact lobes, forming a continuous contour, according to embodiments of the present disclosure. Contact lobes 261, 262, 263, and 264 may form a continuous contour.

In yet other words, contact lobes that may form a continuous contour may be described as merged into a single area. It is noted, however, that merged contact lobes may include contact lobes forming a discontinuous contour, as described hereinbelow.

Some elements and settings illustrated in FIG. 2C may be similar to those illustrated in FIG. 2A. Therefore, for brevity, reference numerals of elements already referred to in describing FIG. 2A are not shown in FIG. 2C.

In embodiments where the contact lobes may form a continuous contour, the contact lobes may include a plurality of intersection regions. The intersection regions may distinguish between contact lobes. Reference is made to FIG. 2D, schematically illustrating intersection regions. Between contact lobes 2261 and 2262 an intersection region 2265a may form. Between contact lobes 2262 and 2263 an intersection region 2265b may form. Intersection region 2265a may include an apex 2266a. Intersection region 2265b may include an apex 2266b. An apex may be a sharp change in a direction of the continuous contour formed by contact lobes, for example, a notched saddle or a pointed dip. In another example, an apex may be a point where a parametrization of coordinates of points of the contour formed by contact lobes, as a function of some parameter, may admit a discontinuous derivative. In some embodiments, a derivative of a parametrization of any coordinate of points of the contour may have a magnitude (absolute value) greater than a predefined threshold. An apex may aid distinguishing between contact lobes.

In some embodiments, the presence of an apex may depend on measured impedance. For example, if the measured impedance of adjacent electrodes is above a predefined threshold, the contact lobes corresponding to the adjacent electrodes may form an apex at the intersection region.

It is noted that rendering an apex may not require computing derivatives. For example, intersection points between contact lobes may be computed. Contact lobes may be delimited by the intersection points. In other words, sections of the contact lobes (e.g., section 2267a, illustrated in dashed lines) may be removed.

In some embodiments, delimitation lines may be included in intersection regions in order to aid distinguishing between contact lobes, for example, delimitation line 2267b included in intersection region 2265b. It is noted that the feature of delimitation line may be independent from the feature of an apex.

Reference is made to FIG. 2E, schematically illustrating yet other exemplary contact lobes according to embodiments of the present disclosure. Contact lobes may include an infill 270, in other words, a color shading of an area region extending between a rendering of the contact lobes and rendering of the catheter distal assembly. In some embodiments, an infill region may extend between a rendering of the contact lobes and a rendering of the organ. The infill may be transparent. That is, the color shading may not conceal visual features where the infill is applied. For example, if an infill is applied on a region including a part of rendering of the organ, the structure of the part of the organ may be visible. In some embodiments, the color of the infill may be the same as a color of the contact lobes. In some embodiments, the color of the infill may be gradient. In other words, the color of the infill may gradually change. It is noted that the feature of infill may be independent from the feature of contact lobes forming a continuous contour.

Reference is made to FIG. 2F, schematically illustrating a variable coloring of contact lobes. In some embodiments, a color of a contact lobe may depend on a measured impedance value of a corresponding electrode. For example, contact lobe 282 may be colored by a first color (illustrated as a dashed line), whereas contact lobe 283 may be colored by a second color (illustrated as a continuous line). The coloring of the contact lobes 282 and 283 may indicate a contact level being above or below a certain threshold. In other words, a first color may indicate the contact level being lower than a minimum threshold, and a second color may indicate the contact level being greater than the minimum threshold.

Some elements and settings illustrated in FIGS. 2E-2F may be similar to those illustrated in FIG. 2C. Therefore, for brevity, reference numerals of elements already referred to in describing FIG. 2C are not shown in FIGS. 2E-2F.

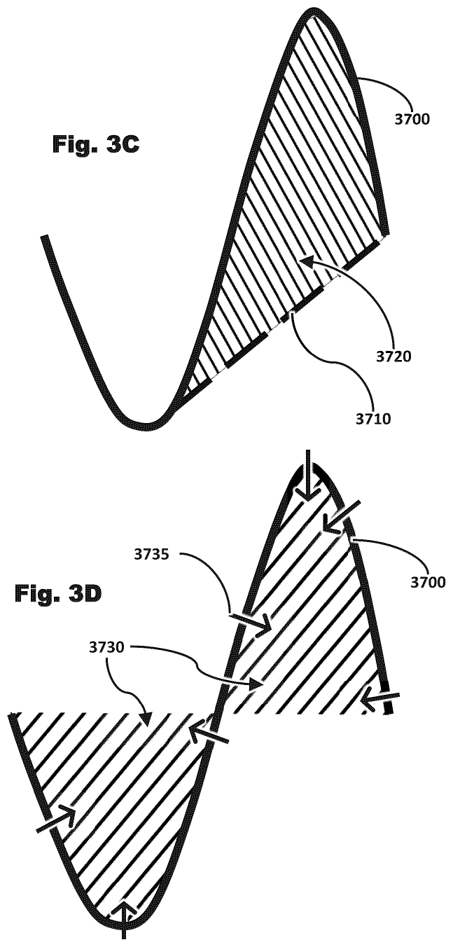

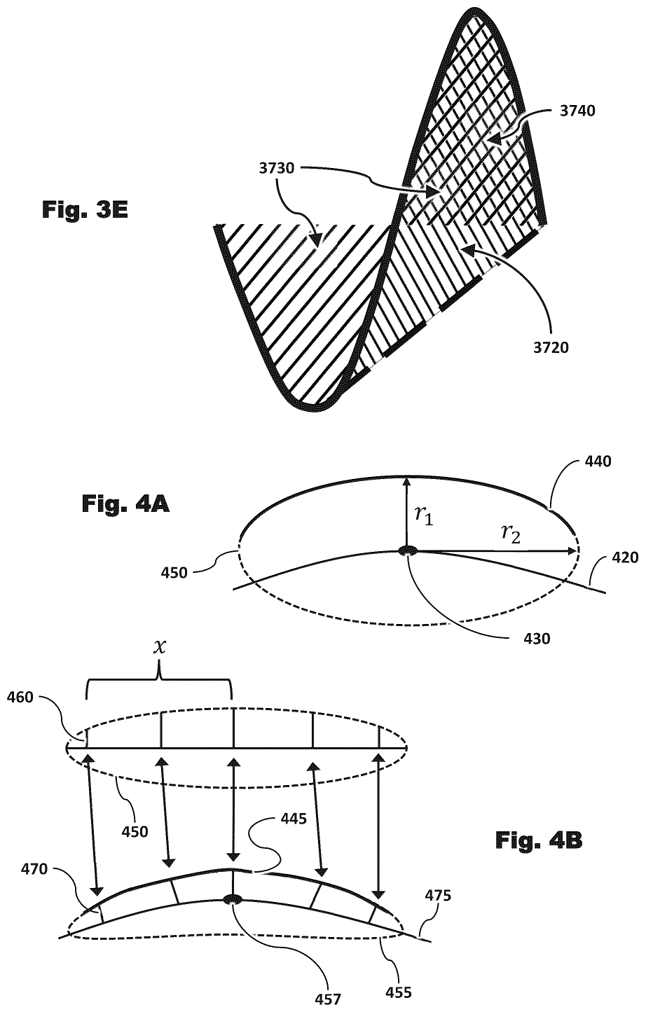

Reference is made to FIGS. 3A-3B, schematically illustrating merging of non-adjacent contact lobes, according to embodiments of the present disclosure. A catheter distal assembly 320 that may be positioned inside an intraluminal organ 310 may be rendered on a display. The catheter distal assembly 320 may include a plurality of electrodes 331, 332, and 333. Contact lobes corresponding to electrodes 331 and 333 may be non-adjacent. That is, the corresponding electrodes 331 and 333 may not be nearest neighbors in the array of electrodes, or may not be adjacent in the linear array of electrodes. For each electrode, a corresponding contact lobe is rendered. That is, contact lobe 341 may correspond to electrode 331, contact lobe 342 may correspond to electrode 332, and contact lobe 343 may correspond to electrode 233. The contact lobes may be overlaid on the graphical representation of the catheter distal assembly 320.

Contact lobes corresponding to electrodes 331 333 may be merged. It is noted that merging of non-adjacent contact lobes may result in a discontinuous contour. This may contrast merging of adjacent contact lobes described herein above. For example, the contour of contact lobe 341 may be split into two discontinuous segments 341a and 341b, and the contour of contact lobe 343 may be split into two discontinuous segments 343a and 343b. An intersection region 365 may include apex 367a formed between segments 341a and 343a, and apex 367b formed between segments 341b and 343b.

Merging of non-adjacent contact lobes may be required, for example, if the catheter distal assembly 320 is sharply bent, e.g., the catheter distal assembly 320 may have a “U-turn”.

It is noted that in FIG. 2A, the contact lobes are “outside” the catheter distal assembly, while in FIG. 3A, they are “inside” the catheter distal assembly. In other words, in FIG. 2A, the contact lobes are positioned in an outer side of the catheter distal assembly, while in FIG. 3A, the contact lobes are positioned in an inner side of the catheter distal assembly. Reference is made to FIGS. 3C-3E, schematically illustrating an inner side according to embodiments of the present disclosure. An “inner side” and an “outer side” of a segment of the catheter distal assembly 3700 can be defined in two ways. An outer side may be defined as any area that is not included in the inner side.

Reference is made to FIG. 3C schematically illustrating an inner side according to a first definition. According to the first definition, the inner side may be an area 3720 delimited by a chord line 3710 between two points on the segment, and the catheter distal assembly 3700. Area 3720 is marked with down-going diagonal stripes.

Reference is made to FIG. 3D schematically illustrating an inner side according to a second definition. According to the second definition, the inner side 3730 may be where displacement, along the transverse direction, forms an acute angle with a local curvature vector, such as local curvature vector 3735. Area 3730 is marked with up-going diagonal stripes.

It is noted that the two definitions hereinabove (of an inner side) may not necessarily be mutually exclusive. As illustrated in FIG. 3E, an area 3740 may be included in the inner side according to both the first definition and the second definition. In other words, area 3740 may be included in area 3720 and in area 3730. Area 3740 is marked with crossing diagonal lines. In other words, area 3740 is marked with both down-going diagonal stripes (marking area 3720) and up-going diagonal stripes (marking area 3730).

Depending on the segment, either of these definitions can be used to define the inner and outer sides of the catheter distal assembly. In some embodiments, methods disclosed herein (e.g., method 100) may include selecting a definition of an inner side, for at least one segment of the catheter distal assembly. In some embodiments, selecting a definition of an inner side may be performed dynamically, i.e., according to a computation performed in real-time, based on measured positions of electrodes included in the catheter distal assembly.

In some embodiments, contact lobes may be positioned exclusively outside the catheter distal assembly. The contact lobes may not necessarily indicate the side of the organ in contact with the electrodes. In embodiments where contact lobes expand asymmetrically relative to the catheter's central axis, they may expand towards the outer side. Placing the contact lobes outside, or expanding them asymmetrically outward, may allow for larger contact lobes, improving the visualization of the contact level.

Reference is made to FIG. 4A, schematically illustrating transverse and tangential radii, according to embodiments of the present disclosure. A catheter distal assembly 420 may have an electrode 430. The catheter distal assembly 420, the electrode 430, and a contact lobe 440 may be rendered on a display. The contact lobe 440 may be an ellipsoidal segment. In other words, contact lobe 440 may have a geometry of an ellipsoidal arc. In other words, contact lobe 440 may be a line segment of an ellipse 450. The ellipse 450 may not be rendered on the display. The contact lobe 440 may be oriented along a tangent direction of the catheter distal assembly. In other words, the ellipse 450 may have two axes: a major axis and a minor axis. One axis (e.g., the major axis) may be parallel to the tangential direction. The other axis (e.g., the minor axis) may be parallel to the transverse direction. A half of the axis parallel to the tangential direction may be referred to as a “tangential radius”. The tangential radius may extend along the tangential direction. The tangential radius may be of length r₁. A half of the axis parallel to the transverse direction may be referred to as a “transverse radius”. The transverse radius may extend along the transverse direction. The transverse radius may be of length r₂. Similarly, the axis parallel to the tangential direction may be referred to as a “tangential diameter”, and the axis parallel to the transversal direction may be referred to as a “transversal diameter”.

In some embodiments, the tangential radius may be constant. In some embodiments, the tangential radius may be based on a spacing between the electrodes of the electrode array. In some embodiments, the tangential radius may be equal to half a sum of distances to adjacent electrodes along the catheter distal assembly. In other words, the tangential radius may be equal to an average separation of a corresponding electrode to nearby electrodes, where the separation may be measured along the catheter distal assembly.

Reference is made to FIG. 4B, schematically illustrating bending of contact lobes, according to embodiments of the present disclosure. In some embodiments, where the contact lobes may include ellipsoidal segments, some of the ellipsoidal segments may be bent according to a curviness of the catheter distal assembly 475. A contact lobe 445 may have a shape of a bent ellipsoidal segment. Each point of the contact lobe 445 may have a distance 470 from the catheter distal assembly 475 that may be equal to a distance 460 between a tangential radius of a regular (unbent) ellipse 450 and a corresponding point on the rim of the ellipse. In other words, the tangential diameter of the ellipse 450 may be bent according to the curviness of the catheter distal assembly 475, while a local half-thickness of the ellipse 450 may be kept. In yet other words, a point on the ellipse 450 may have a distance of r₁\sqrt{(x/r₂)²−1} from the tangent diameter, where x may denote a distance along the tangent diameter from the center of the ellipse 450. A point on a bent ellipse 455 may have a distance of r₁\sqrt{(y/r₂)²−1} from the catheter distal assembly 475, where y may denote a distance along the catheter distal assembly from a center point. In some embodiments, the center point may be an electrode 457. In other words, in some embodiments, a point on a bent ellipse 455 may have a distance of r₁\sqrt{(y/r₂)²−1} from the catheter distal assembly 475, where y may denote a distance along the catheter distal assembly from a corresponding electrode 457. This distance may be referred to as a “local radius”.

Reference is made to FIG. 1A. The step 120 of dynamically updating visual features may include a step 125 of computing a size of the contact lobes. In some embodiments, where contact lobes may include ellipsoidal segments, the step 125 of computing a size of the contact lobes may include a step 130 of computing the radius of the ellipsoidal segments (e.g., tangential radius and transverse radius). In embodiments where a transverse radius may be computed, the transverse radius may be variable. The transverse radius may be based on the contact level between the tissue wall and an electrode. As indicated hereinabove, the transverse radius may extend in a direction transverse to a local orientation of the catheter distal assembly around the (corresponding) electrode.

In some embodiments, the variable transverse radius may vary within a radius adjustment range bounded by a minimum radius threshold value and a maximum radius threshold value. In other words, the variable transverse radius may have a minimum value and a maximum value. In some embodiments, the minimum radius threshold may correspond to a zero-contact level. In other words, the minimum radius threshold may correspond to a state of no-contact level between the tissue wall and the electrode.

In some embodiments, the step 120 of dynamically updating visual features may include a step 140 of determining a color of the contact lobes (e.g., according to a contact level, as described hereinabove).

In some embodiments, the step 120 of dynamically updating visual features may include a step 150 of computing orientation of the contact lobes. For example, as indicated hereinabove, an orientation of a contact lobe may be along a tangential direction. It is noted, however, that orientation of contact lobes may not be limited to the transverse direction. The step 150 of computing orientations of contact lobes may include, for example, computing line-parameters of a lines defining a longitudinal extent of the contact lobes.

In some embodiments, the step 120 of dynamically updating visual features may include a step 160 of merging contact lobes. In some embodiments, the step 160 of merging contact lobes may include a step 165 of computing intersection points between contact lobes.

In some embodiments, the step 160 of merging contact lobes may include a step 170 of computing an apex between contact lobes. The step 170 of computing an apex may include determining whether an apex may be included. In some embodiments, the step 170 of computing an apex may include comparing a transverse radius to an adjacent transverse radius of an adjacent electrode. If a difference between the transverse radius and the adjacent transverse radius is lower than a predefined comparison threshold, a local transverse radius at an intersection point may be lower than the transverse radius by a predefined dip threshold. In other words, an intersection point may be closer to the catheter distal assembly than the transverse radius by at least a predefined threshold.

In some embodiments, the step 120 of dynamically updating visual features may include a step 175 of computing local transverse radii. The step 175 of computing local transverse radii may include computing for each contact lobe, a plurality of local transverse radius at a corresponding plurality of positions according to a tangential distance from the electrode. In other words, distances from the catheter distal assembly, or from a reference axis (e.g., an axis defining a longitudinal extent or an axis of an ellipse), may be computed. In some embodiments, a local radius is computed for distance increments being 0.5 millimeters. The distance increments may be distance increments along the catheter distal assembly.

In some embodiments, the step 120 of dynamically updating visual features may include a step 180 of computing vertices. In other words, coordinates of points of the contact lobes may be computed. In some embodiments, the vertices may be vertices of polygons approximating the exact shape of the contact lobes. Polygons approximating the exact shape of the contact lobes may be required, for example, if a computer implementing the method 100 may require curves to be approximated by a plurality of straight lines.

In some embodiments, the step 120 of dynamically updating visual features may include a step 185 of computing an infill. The step 185 of computing an infill may include determining whether an infill is to be included. If an infill is included, the step 185 of computing an infill may include computing a desired color of the infill. The step 185 of computing an infill may include computing a transparency of the infill, e.g., an alpha channel of an RGBA color format.

In some embodiments, the step 120 of dynamically updating visual features may include a step 190 of providing computation results to a graphics engine, in order to render the contact lobes. In some embodiments, some of the computations may be performed by the graphics engine, for example the step 180 of computing vertices.

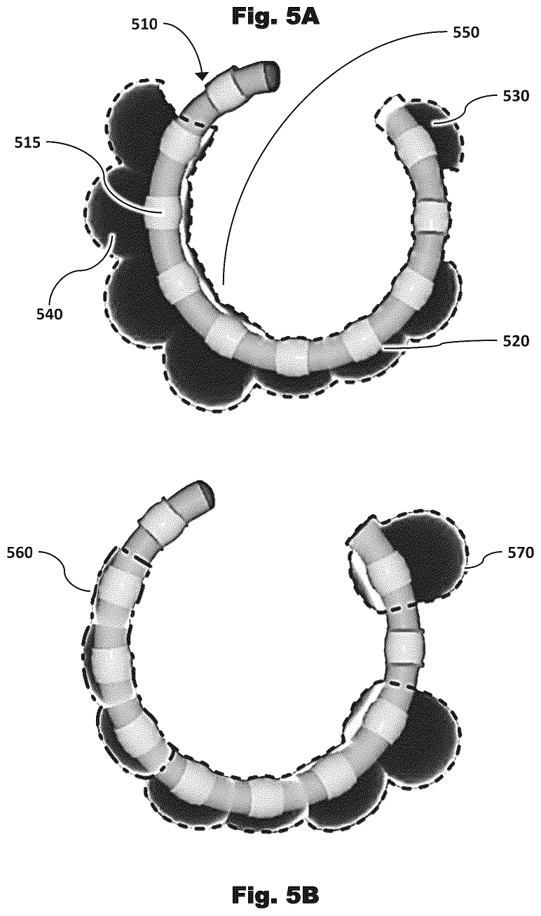

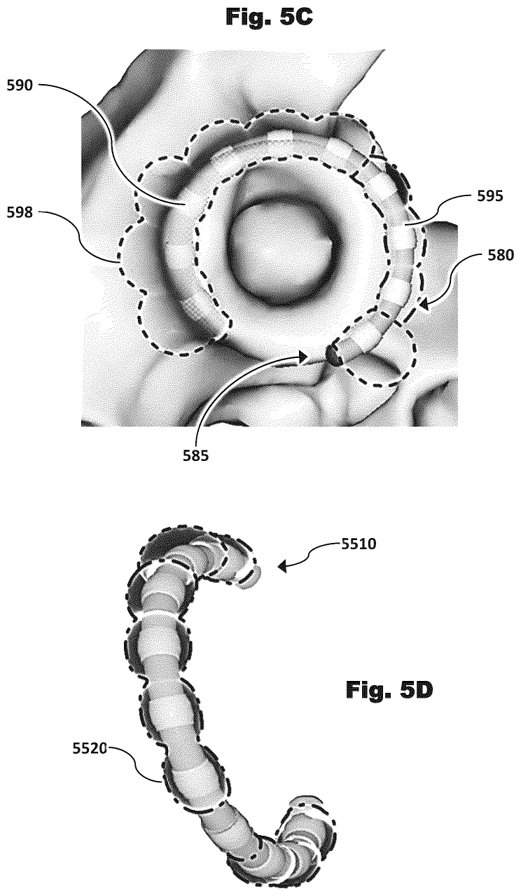

Reference is made to FIG. 5A, showing an exemplary screenshot of visual output of methods and systems according to embodiments of the present disclosure. A catheter distal assembly 510 comprising a plurality of electrodes (such as electrode 515) is rendered along with a plurality of contact lobes such as contact lobes 520, 530, 540, and 550. The contact lobes are rendered with dashed lines. The contact lobes expand asymmetrically with respect to a central axis of the catheter distal assembly, so as to predominantly extend in a transverse direction towards the tissue wall. The contact lobes 520, 530, 540, and 550 are ellipsoidal segments. Some of the contact lobes form a single contour. The contact lobes include an infill.

Reference is made to FIG. 5B, showing another exemplary screenshot of visual output of methods and systems according to embodiments of the present disclosure. Contact lobes are rendered with dashed lines. Contact lobe 560 has one color, whereas contact lobe 570 has a second color. The different colors are shown as different types of dashes. Contact lobe 560 is an ellipsoidal segment, whereas contact lobe 570 is not. Contact lobe 560 includes an infill, whereas contact lobe 570 does not. Contact lobe 560 has a minimum transverse radius. Some of the contact lobes form a single contour.

Reference is made to FIG. 5C, showing yet another exemplary screenshot of visual output of methods and systems according to embodiments of the present disclosure. A catheter distal assembly 585 comprising a plurality of electrodes (such as electrode 590) is rendered along with a plurality of contact lobes such as contact lobes 595 and 598. Contact lobes are rendered with dashed lines. An organ 580 is also rendered. The contact lobes expand asymmetrically with respect to a central axis of the catheter distal assembly, so as to predominantly extend in a transverse direction towards the tissue wall. Contact lobe 595 has a minimum transverse radius. Contact lobe 595 has one color, whereas contact lobe 598 has a second color. The different colors are shown as different types of dashes. All of the contact lobes form a single contour.

Reference is made to FIG. 5D, showing yet another exemplary screenshot of visual output of methods and systems according to embodiments of the present disclosure. A catheter distal assembly 5510 comprising a plurality of electrodes is rendered along with a plurality of contact lobes such as contact lobe 5520. The viewing position of the catheter distal assembly 5510 in FIG. 5D is from the side, whereas the viewing position of catheter distal assemblies in FIGS. 5A-5C is from the top. It is noted that the contact lobes may be three-dimensional objects, where each contact lobe may include a plurality of line segments (e.g., ellipsoidal segments) that may be rendered on a display.

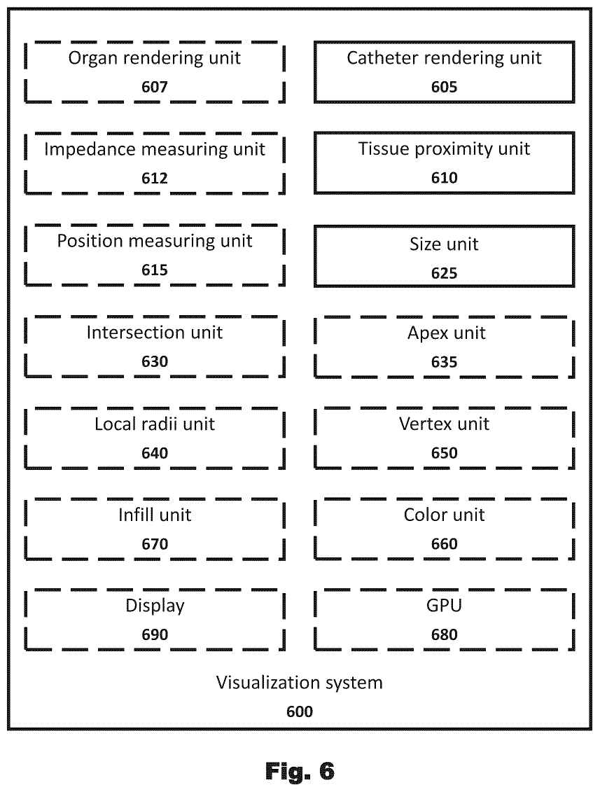

Reference is made to FIG. 6 which shows a block diagram schematically illustrating a system according to embodiments of the present disclosure. A visualization system 600 may be configured to implement methods according to the present disclosure (e.g., method 100 schematically illustrated in FIG. 1B) in order to provide, in real-time, visual feedback of a contact level between a tissue wall of a luminal organ and an ablation catheter. The system 600 may be, for example, a computer system.

The visualization system 600 may include a catheter rendering unit 605, in order to render the catheter distal assembly. The visualization system 600 may include an organ rendering unit 607, in order to render the organ in which the catheter distal assembly is positioned. The visualization system 600 may include a tissue proximity unit 610, in order to assess proximity (contact level) of electrodes to the tissue wall. The visualization system 600 may include an impedance measuring unit 612, in order to measure impedance of at least one electrode included in the catheter distal assembly. The visualization system 600 may include a position measuring unit 615, in order to measure a position of the catheter distal assembly. The visualization system 600 may include a size unit 625, in order to compute the size of contact lobes (e.g., transverse radius). The visualization system 600 may include an intersection unit 630, in order to compute intersection points between contact lobes. The visualization system 600 may include an apex unit 635, in order to determine whether an apex may be required at an intersection region between contact lobes, and compute parameters of the apex. The visualization system 600 may include a local radii unit 640, in order to compute local radii (a plurality of local radius) of points of contact lobes. The visualization system 600 may include a vertex unit 650, in order to compute coordinates of points of the contact lobes. The visualization system 600 may include a color unit 660, in order to determine a color of the contact lobes. The visualization system 600 may include an infill unit 670, in order to determine whether an infill is included in a contact lobe. The visualization system 600 may include a GPU 680, in order to efficiently perform rendering computations. The visualization system 600 may include a display 690, in order to display the visual feedback.

### SUMMARY

Following is a non-exclusive list of some exemplary examples of the disclosure. The present disclosure also includes examples which include fewer than all the features in an example, and examples using features from multiple examples, even if not listed below.

### Example 1

A computer-implemented method of providing real-time visual feedback of a contact level between a tissue wall of a luminal organ and an ablation catheter. The ablation catheter comprising a plurality of electrodes forming an elongated electrode array placed along a catheter distal assembly. The method comprising, while the catheter distal assembly is in the luminal organ of a patient, rendering on a display a graphical representation of the catheter distal assembly and the plurality of electrodes thereon. The method comprising, while the catheter distal assembly is in the luminal organ of a patient, for each electrode of the plurality of electrodes, repeatedly assessing tissue proximity of each of the plurality of electrodes. Further, for each electrode of the plurality of electrodes, the method comprising dynamically updating visual features indicative of the tissue proximity. The visual features comprising contact lobes, wherein each contact lobe is centered on a corresponding electrode and overlaid on said graphical representation of the catheter distal assembly, and a size of said contact lobe increases with the contact level between said tissue wall and said corresponding electrode, based on the tissue proximity.

### Example 2

The computer-implemented method according to example 1, wherein assessing tissue proximity comprises measuring impedance of at least one electrode.

### Example 3

The computer-implemented method according to any one of examples 1 to 2, wherein when the tissue proximity of adjacent electrodes is above a predefined threshold, the contact lobes corresponding to said adjacent electrodes form an apex at an intersection region.

### Example 4

The computer-implemented method according to any one of examples 1 to 3, wherein said contact lobes are configured to expand asymmetrically with respect to a central axis of the catheter, so as to predominantly extend in a transverse direction towards the tissue wall.

### Example 5

The computer-implemented method according to any one of examples 3 to 4, comprising merging overlapping contact lobes, corresponding to distinct electrodes, into a single area.

### Example 6

The computer-implemented method according to any one of examples 1 to 5, wherein said contact lobes comprise ellipsoidal segments.

### Example 7

The computer-implemented method according to example 6, comprising, for each electrode, computing a variable transverse radius of said ellipsoidal segments, said variable transverse radius being based on the contact level between the tissue wall and said electrode and extending in a direction transverse to a local orientation of the catheter around said electrode.

### Example 8

The computer-implemented method according to example 7, comprising comparing a transverse radius to an adjacent transverse radius of an adjacent electrode. Wherein, when a difference between the transverse radius and the adjacent transverse radius is lower than a predefined comparison threshold, a local transverse radius at an intersection point is lower than the transverse radius by a predefined dip threshold.

### Example 9

The computer-implemented method according to any one of examples 7 to 8, wherein said variable transverse radius varies within a radius adjustment range bounded by a minimum radius threshold value and a maximum radius threshold value.

### Example 10

The computer-implemented method according to example 9, wherein said minimum radius threshold corresponds a zero-contact level.

### Example 11

The computer-implemented method according to any one of examples 6 to 10, wherein said ellipsoidal segments include a tangential radius extending in a direction of the local orientation of said catheter.

### Example 12

The computer-implemented method according to example 11, wherein said tangential radius is constant and based on a spacing between the electrodes of the electrode array.

### Example 13

The computer-implemented method according to any one of examples 11 to 12, wherein the tangential radius equals half a sum of distances to adjacent electrodes along the catheter.

### Example 14

The computer-implemented method according to any one of examples 11 to 13, wherein the local orientation of the catheter around a given electrode is defined according to a line tangent to said catheter distal assembly at a location of the given electrode.

### Example 15

The computer-implemented method according to any one of examples 3 to 14, comprising merging overlapping contact lobes, corresponding to distinct electrodes, into a single arca.

### Example 16

The computer-implemented method according to any one of examples 1 to 15, comprising coloring the contact lobes using a transparent infill.

### Example 17

The computer-implemented method according to example 16, wherein a first color indicates the contact level being lower than a minimum threshold, and a second color indicates the contact level being greater than the minimum threshold.

### Example 18

The computer-implemented method according to any one of examples 6 to 17, wherein said ellipsoidal segments are bent according to a curviness of the catheter.

### Example 19

The computer-implemented method according to any one of examples 1 to 18, comprising computing for each contact lobe, a plurality of local transverse radius at a corresponding plurality of positions at according to a tangential distance from the electrode.

### Example 20

The computer-implemented method according to example 19, wherein said local radius is computed for distance increments being 0.5 millimeter.

### Example 21

The computer-implemented method according to any one of examples 1 to 20, wherein the luminal organ is a heart of the patient.

### Example 22

The computer-implemented method according to any one of examples 1 to 21, wherein all electrodes included in the plurality of electrodes forms a linear array.

### Example 23

The computer-implemented method according to any one of examples 1 to 22, comprising rendering on the display an anatomy of the luminal organ.

### Example 24

A graphical user interface (GUI) for providing real-time visual feedback of contact level between a tissue wall of a luminal organ and an ablation catheter. The ablation catheter comprising a plurality of electrodes forming an elongated electrode array placed along a catheter distal assembly. The GUI being executable by a computer to, while the catheter distal assembly is in the luminal organ of a patient, rendering on a display a graphical representation of the catheter distal assembly and the plurality of electrodes thereon. The GUI being executable by a computer to, while the catheter distal assembly is in the luminal organ of a patient, for each electrode of the plurality of electrodes, repeatedly receive tissue proximity values of each of the plurality of electrodes. Further, for each electrode of the plurality of electrodes, the GUI being executable by a computer to dynamically update visual features indicative of a tissue proximity value, responsive to a change detected in the tissue proximity value. The visual features comprising contact lobes, wherein each contact lobe is centered on a corresponding electrode and overlaid on said graphical representation of the catheter distal assembly, and a size of said contact lobe increases with the contact level between said tissue wall and said corresponding electrode, based on the tissue proximity value.

### Example 25

The GUI according to example 24, wherein the tissue proximity values correspond measured impedance values of the plurality of electrodes.

### Example 26

The GUI according to any one of examples 24 to 25, wherein when the measured impedance of adjacent electrodes is above a predefined threshold, the contact lobes corresponding to said adjacent electrodes form an apex at an intersection region.

### Example 27

The GUI according to any one of examples 24 to 26, wherein said contact lobes are configured to expand asymmetrically with respect to a central axis of the catheter, so as to predominantly extend in a transverse direction towards the tissue wall.

### Example 28

The GUI according to any one of examples 26 to 27, comprising merging overlapping contact lobes, corresponding to distinct electrodes, into a single area.

### Example 29

The GUI according to any one of examples 24 to 28, wherein said contact lobes comprise ellipsoidal segments.

### Example 30

The GUI according to example 29, comprising, for each electrode, computing a variable transverse radius of said ellipsoidal segments, said variable transverse radius being based on the contact level between the tissue wall and said electrode and extending in a direction transverse to a local orientation of the catheter around said electrode.

### Example 31

The GUI according to example 30, comprising comparing a transverse radius to an adjacent transverse radius of an adjacent electrode. Wherein, when a difference between the transverse radius and the adjacent transverse radius is lower than a predefined comparison threshold, a local transverse radius at an intersection point is lower than the transverse radius by a predefined dip threshold.

### Example 32

The GUI according to any one of examples 30 to 31, wherein said variable transverse radius varies within a radius adjustment range bounded by a minimum radius threshold value and a maximum radius threshold value.

### Example 33

The GUI according to example 32, wherein said minimum radius threshold corresponds a zero-contact level.

### Example 34

The GUI according to any one of examples 29 to 33, wherein said ellipsoidal segments include a tangential radius extending in a direction of the local orientation of said catheter.

### Example 35

The GUI according to example 34, wherein said tangential radius is constant and based on a spacing between the electrodes of the electrode array.

### Example 36

The GUI according to any one of examples 34 to 35, wherein the tangential radius equals half a sum of distances to adjacent electrodes along the catheter.

### Example 37

The GUI according to any one of examples 34 to 36, wherein the local orientation of the catheter around a given electrode is defined according to a line tangent to said catheter distal assembly at a location of the given electrode.

### Example 38

The GUI according to any one of examples 26 to 37, comprising merging overlapping contact lobes, corresponding to distinct electrodes, into a single area.

### Example 39

The GUI according to any one of examples 24 to 38, comprising coloring the contact lobes using a transparent infill.

### Example 40

The GUI according to example 39, wherein a first color indicates the contact level being lower than a minimum threshold, and a second color indicates the contact level being greater than the minimum threshold.

### Example 41

The GUI according to any one of examples 29 to 40, wherein said ellipsoidal segments are bent according to a curviness of the catheter.

### Example 42

The GUI according to any one of examples 24 to 41, comprising computing for each contact lobe, a plurality of local transverse radius at a corresponding plurality of positions at according to a tangential distance from the electrode.

### Example 43

The GUI according to example 42, wherein said local radius is computed for distance increments being 0.5 millimeter.

### Example 44

The GUI according to any one of examples 24 to 43, wherein the luminal organ is a heart of the patient.

### Example 45

The GUI according to any one of examples 24 to 44, wherein all electrodes included in the plurality of electrodes forms a linear array.

### Example 46

The GUI according to any one of examples 24 to 45, comprising rendering on the display an anatomy of the luminal organ.

### Example 47

A computer system comprising at least one processing circuitry, configured to execute a method of providing real-time visual feedback of a contact level between a tissue wall of a luminal organ and an ablation catheter. The ablation catheter comprising a plurality of electrodes forming an elongated electrode array placed along a catheter distal assembly. The method comprising, while the catheter distal assembly is in the luminal organ of a patient, rendering on a display a graphical representation of the catheter distal assembly and the plurality of electrodes thereon. The method comprising, while the catheter distal assembly is in the luminal organ of a patient, for each electrode of the plurality of electrodes, repeatedly assessing tissue proximity of each of the plurality of electrodes. Further, for each electrode of the plurality of electrodes, the method comprising dynamically updating visual features indicative of the tissue proximity. The visual features comprising contact lobes, wherein each contact lobe is centered on a corresponding electrode and overlaid on said graphical representation of the catheter distal assembly, and a size of said contact lobe increases with the contact level between said tissue wall and said corresponding electrode, based on the tissue proximity.

### Example 48

The computer system according to example 47, wherein assessing tissue proximity comprises measuring impedance of at least one electrode.

### Example 49

The computer system according to any one of example 47 to 48, wherein when the tissue proximity of adjacent electrodes is above a predefined threshold, the contact lobes corresponding to said adjacent electrodes form an apex at an intersection region.

### Example 50

The computer system according to any one of examples 47 to 49, wherein said contact lobes are configured to expand asymmetrically with respect to a central axis of the catheter, so as to predominantly extend in a transverse direction towards the tissue wall.

### Example 51

The computer system according to any one of examples 49 to 50, comprising merging overlapping contact lobes, corresponding to distinct electrodes, into a single area.

### Example 52

The computer system according to any one of examples 47 to 51, wherein said contact lobes comprise ellipsoidal segments.

### Example 53

The computer system according to example 52, comprising, for each electrode, computing a variable transverse radius of said ellipsoidal segments, said variable transverse radius being based on the contact level between the tissue wall and said electrode and extending in a direction transverse to a local orientation of the catheter around said electrode.

### Example 54

The computer system according to example 53, comprising comparing a transverse radius to an adjacent transverse radius of an adjacent electrode. Wherein, when a difference between the transverse radius and the adjacent transverse radius is lower than a predefined comparison threshold, a local transverse radius at an intersection point is lower than the transverse radius by a predefined dip threshold.

### Example 55

The computer system according to any one of examples 53 to 54, wherein said variable transverse radius varies within a radius adjustment range bounded by a minimum radius threshold value and a maximum radius threshold value.

### Example 56

The computer system according to example 55, wherein said minimum radius threshold corresponds a zero-contact level.

### Example 57

The computer system according to any one of examples 52 to 55, wherein said ellipsoidal segments include a tangential radius extending in a direction of the local orientation of said catheter.

### Example 58

The computer system according to example 57, wherein said tangential radius is constant and based on a spacing between the electrodes of the electrode array.

### Example 59

The computer system according to any one of examples 57 to 58, wherein the tangential radius equals half a sum of distances to adjacent electrodes along the catheter.

### Example 60

The computer system according to any one of examples 57 to 59, wherein the local orientation of the catheter around a given electrode is defined according to a line tangent to said catheter distal assembly at a location of the given electrode.

### Example 61

The computer system according to any one of examples 49 to 60, comprising merging overlapping contact lobes, corresponding to distinct electrodes, into a single area.

### Example 62

The computer system according to any one of examples 47 to 61, comprising coloring the contact lobes using a transparent infill.

### Example 63

The computer system according to example 62, wherein a first color indicates the contact level being lower than a minimum threshold, and a second color indicates the contact level being greater than the minimum threshold.

### Example 64

The computer system according to any one of examples 62 to 63, wherein said ellipsoidal segments are bent according to a curviness of the catheter.

### Example 65

The computer system according to any one of examples 47 to 64, comprising computing for each contact lobe, a plurality of local transverse radius at a corresponding plurality of positions at according to a tangential distance from the electrode.

### Example 66

The computer system according to example 65, wherein said local radius is computed for distance increments being 0.5 millimeter.

### Example 67

The computer system according to any one of examples 47 to 66, wherein the luminal organ is a heart of the patient.

### Example 68

The computer system according to any one of examples 47 to 67, wherein all electrodes included in the plurality of electrodes forms a linear array.

### Example 69

The computer system according to any one of examples 47 to 68, comprising rendering on the display an anatomy of the luminal organ.

### Example 70

A non-transitory computer readable storage medium tangibly embodying a program of instructions that, when executed by a computer, cause the computer to perform a method according to any one of examples 1 to 23.

### Example 71

A non-transitory computer readable storage medium tangibly embodying a program of instructions that, when executed by a computer, cause the computer to execute a GUI according to any one of examples 24 to 46.

Those skilled in the art to which the present disclosure pertains, can appreciate that while the present disclosure has been described in terms of preferred examples, the concept upon which this disclosure is based may readily be utilized as a basis for the designing of other structures, systems, and processes for carrying out the several purposes of the present disclosure.

Also, it is to be understood that the phraseology and terminology employed herein are for the purpose of description and should not be regarded as limiting. It should be noted that the words “comprising”, “including” and “having” as used throughout the appended claims are to be interpreted to mean “including but not limited to”. The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases, and disjunctively present in other cases. The term “each” may not be exclusively understood as referring to each and every, and, when technically relevant, may also refer to “at least some”.

All patents and patent applications mentioned in this specification are herein incorporated in their entirety by reference into the specification, to the same extent as if each individual patent or patent application was specifically and individually indicated to be incorporated herein by reference. In addition, citation or identification of any reference in this application shall not be construed as an admission that such reference is available as prior art to the present disclosure.

It is important, therefore, that the scope of the present disclosure is not construed as being limited by the illustrative examples set forth herein. Other variations are possible within the scope of the present disclosure as defined in the appended claims. Other combinations and sub-combinations of features, functions, elements and/or properties, may be claimed through amendment of the present claims or presentation of new claims in this or a related application. Such amended or new claims, whether they are directed to different combinations or directed to the same combinations, whether different, broader, narrower, or equal in scope to the original claims, are also regarded as included within the subject matter of the present description.

1. A computer-implemented method of providing real-time visual feedback of a contact level between a tissue wall of a luminal organ and an ablation catheter comprising a plurality of electrodes forming an elongated electrode array placed along a catheter distal assembly, the method comprising:
while the catheter distal assembly is in the luminal organ of a patient:
(a) rendering on a display a graphical representation of the catheter distal assembly and the plurality of electrodes thereon;
(b) for each electrode of the plurality of electrodes:
i. repeatedly assessing tissue proximity of each of the plurality of electrodes; and
ii. dynamically updating visual features indicative of the tissue proximity, said visual features comprising contact lobes, wherein
each contact lobe is centered on a corresponding electrode and overlaid on said graphical representation of the catheter distal assembly;
a size of said contact lobe increases with the contact level between said tissue wall and said corresponding electrode, based on the tissue proximity.

2. The computer-implemented method of claim 1, wherein assessing tissue proximity comprises measuring impedance of at least one electrode.

3. The computer-implemented method of claim 1, wherein when the assessed tissue proximity of adjacent electrodes is above a predefined threshold, the contact lobes corresponding to said adjacent electrodes form an apex at an intersection region.

4. The computer-implemented method of claim 1, wherein said contact lobes are configured to expand asymmetrically with respect to a central axis of the catheter, so as to predominantly extend in a transverse direction towards the tissue wall.

5. The computer-implemented method of claim 3, comprising merging overlapping contact lobes, corresponding to distinct electrodes, into a single area.

6. The computer-implemented method of claim 1, wherein said contact lobes comprise ellipsoidal segments.

7. The computer-implemented method of claim 6, comprising, for each electrode, computing a variable transverse radius of said ellipsoidal segments, said variable transverse radius being based on the contact level between the tissue wall and said electrode and extending in a direction transverse to a local orientation of the catheter around said electrode.

8. A graphical user interface (GUI) for providing real-time visual feedback of contact level between a tissue wall of a luminal organ and an ablation catheter comprising a plurality of electrodes forming an elongated electrode array placed along a catheter distal assembly, the GUI being executable by a computer to:
while the catheter distal assembly is in the luminal organ of a patient:
(a) render, on a display, a graphical representation of the catheter distal assembly and the plurality of electrodes thereon;
(b) for each electrode of the plurality of electrodes:
i. repeatedly receive tissue proximity values of each of the plurality of electrodes; and
ii. responsive to a change detected in a tissue proximity value, dynamically update visual features indicative of the tissue proximity value, said visual features comprising contact lobes, wherein
each contact lobe is centered on a corresponding electrode and overlaid on said graphical representation of the catheter distal assembly; and
a size of said contact lobe increases with the contact level between said tissue wall and said corresponding electrode, based on the tissue proximity value.

9. The GUI of claim 8, wherein the tissue proximity values correspond measured impedance values of the plurality of electrodes.

10. The GUI of claim 8, wherein when the tissue proximity values of adjacent electrodes is above a predefined threshold, the contact lobes corresponding to said adjacent electrodes form an apex at an intersection region.

11. The GUI of claim 8, wherein said contact lobes are configured to expand asymmetrically with respect to a central axis of the catheter, so as to predominantly extend in a transverse direction towards the tissue wall.

12. The GUI of claim 10, configured to merge overlapping contact lobes, corresponding to distinct electrodes, into a single area.

13. The GUI of claim 8, wherein said contact lobes comprise ellipsoidal segments.

14. The GUI of claim 13, configured to compute, for each electrode, a variable transverse radius of said ellipsoidal segments, said variable transverse radius being based on the contact level between the tissue wall and said electrode and extending in a direction transverse to a local orientation of the catheter around said electrode.

15. A computer system comprising at least one processing circuitry, configured to execute a method of providing real-time visual feedback of a contact level between a tissue wall of a luminal organ and an ablation catheter comprising a plurality of electrodes forming an elongated electrode array placed along a catheter distal assembly, the method comprising:
while the catheter distal assembly is in the luminal organ of a patient:
(a) rendering on a display a graphical representation of the catheter distal assembly and the plurality of electrodes thereon;
(b) for each electrode of the plurality of electrodes:
i. repeatedly assessing tissue proximity of each of the plurality of electrodes; and
ii. dynamically updating visual features indicative of the tissue proximity, said visual features comprising contact lobes, wherein
each contact lobe is centered on a corresponding electrode and overlaid on said graphical representation of the catheter distal assembly;
a size of said contact lobe increases with the contact level between said tissue wall and said corresponding electrode, based on the tissue proximity.

16. The computer system of claim 15, wherein assessing tissue proximity comprises measuring impedance of at least one electrode.

17. The computer system of claim 15, wherein when the tissue proximity of adjacent electrodes is above a predefined threshold, the contact lobes corresponding to said adjacent electrodes form an apex at an intersection region.

18. The computer system of claim 15, wherein said contact lobes are configured to expand asymmetrically with respect to a central axis of the catheter, so as to predominantly extend in a transverse direction towards the tissue wall.

19. The computer system of claim 15, wherein said contact lobes comprise ellipsoidal segments.

20. The computer system of claim 19, wherein the method comprises, for each electrode, computing a variable transverse radius of said ellipsoidal segments, said variable transverse radius being based on the contact level between the tissue wall and said electrode and extending in a direction transverse to a local orientation of the catheter around said electrode.
